(19) United States
(12) Patent Application Publication
(10) Pub. No.: US 2011/0071596 A1
(43) Pub. Date: Mar. 24, 2011

(54) ELECTRODE CONTACTS FOR A MEDICAL IMPLANT

(76) Inventors: Sule Kara, Coogee (AU); Martin Svehla, Botany (AU); James G.E. Smith, Sydney (AU); Edmond D. Capcelea, Bondi Junction (AU)

(21) Appl. No.: 12/842,029

(22) Filed: Jul. 22, 2010

Related U.S. Application Data
(63) Continuation of application No. 12/743,804, filed on Oct. 29, 2010.

(30) Foreign Application Priority Data
Nov. 19, 2007 (AU) 2007906334
Jul. 22, 2009 (AU) 2009903424

(51) Int. Cl. A61F 11/04; A61N 1/36; H01R 43/00; B82Y 5/00

(52) U.S. Cl. 607/57; 607/137; 29/885; 977/742

(57) ABSTRACT

A electrode array for a medical and in particular cochlear implant and a method of manufacturing such an array is described in which carbon nanotubes are deposited onto electrode contacts at temperatures below the service temperature of a carrier material supporting the electrode contacts. This allows the carrier material, which may be a polymer like silicone, to be molded about the electrode contacts before they are coated with carbon nanotubes to allow for the provision of smaller and more highly concentrated electrodes.

### CROSS-REFERENCE TO RELATED APPLICATIONS

This application is a Continuation-in-Part of U.S. application Ser. No. 12/743,804, filed on May 19, 2010, entitled “ELECTRODE ARRAY FOR A COCHLEAR IMPLANT,” which is a National Stage Application of International Application No. PCT/AU2008/001718, filed Nov. 19, 2008, entitled “ELECTRODE ARRAY FOR A COCHLEAR IMPLANT,” which claims priority from Australian Provisional Patent Application No. 2007906334, filed Nov. 19, 2007. The contents of these applications are hereby incorporated by reference herein.

This application also claims priority from Australian Provisional Application No. 2009903424 filed on Jul. 22, 2009. The contents of this application is hereby incorporated by reference herein.

### BACKGROUND

1. Field of the Invention

The present invention relates generally to medical devices, and more particularly, to electrode contacts of medical devices.

2. Related Art

Medical devices are used in many areas of medicine to enhance the length and/or quality of the life of the device recipient. Some medical devices are partially or completely implantable. Such implantable medical devices include, for example, pacemakers, controlled drug delivery implants and cochlear implants.

A cochlear implant applies electrical stimulation signals directly to the auditory nerve fibers of the recipient, allowing the brain to perceive a hearing sensation approximating the natural hearing sensation. These stimulating signals are applied by an array of electrode contacts implanted into the recipient's cochlea.

The electrode array is connected to a stimulator unit, which generates the electrical stimulation signals delivered by the electrode array. The stimulator unit is operationally connected to a sound processing unit. The sound processing unit receives audio signals from one or more microphones or other sound input devices, and processes the audio signals to generate control signals for the stimulator. The sound processing unit and microphone(s) are traditionally located externally to the recipient and the stimulator is implanted within the recipient, usually near the mastoid on the recipient's skull and underneath the surrounding tissue.

The effectiveness of the cochlear implant depends on many factors, including the quality and quantity of the electrode contacts. While medical implants with electrode arrays currently exist that are functional to provide stimulating signals, the effectiveness and number of the electrode contacts is limited by currently used materials and manufacturing techniques for electrode arrays.

### SUMMARY

In one aspect of the present invention, a method of manufacturing an electrode array for a medical implant is disclosed, the method comprising: providing a plurality of spaced apart electrode contacts supported by a biocompatible polymeric carrier material extending around the contacts, leaving at least a portion of each of the plurality of electrode contacts exposed, each electrode contact being connected to a respective conductive path extending from the electrode contact; and depositing carbon nanotubes on the exposed portion of one or more of the plurality of electrode contacts at a temperature less than a service temperature of the polymeric carrier material.

In another aspect, an electrode array for a medical implant is disclosed, the electrode array comprising a plurality of spaced apart electrode contacts that each have an exposed portion on which is deposited carbon nanotubes, each contact being connected to at least one conductive path, and a biocompatible carrier material supporting the conductive paths and electrode contacts and extending around the exposed portions of the electrode contacts.

In a further aspect of the present invention, a medical implant system is disclosed, the system comprising: an external component for receiving an input signal and for processing the input signal to provide a control signal; an internal component for receiving the control signal and for converting the control signal into a stimulation signal and applying the stimulation signal to tissue of an implantee via a plurality of spaced apart electrode contacts connected to the internal component via a plurality of corresponding conductive paths, with a carrier material extending between and supporting each of the electrode contacts, wherein each electrode contact has an exposed portion on which is deposited carbon nanotubes.

### BRIEF DESCRIPTION OF THE DRAWINGS

Illustrative embodiments of the present invention are described herein with reference to the accompanying figures, in which:

FIG. 1—is a side view of an exemplary cochlear implant having an array of electrode contacts, in accordance with embodiments of the present invention;

FIG. 1A is an enlarged side view of the electrode array illustrated in FIG. 1;

FIG. 2 is a high-level flow chart of the primary operations which may be implemented to form an electrode array according to an aspect of the present invention;

FIG. 3 is a side view of a cochlear implant including a stimulator with an electrode array as shown in FIG. 1A;

FIG. 4 is a perspective view of a process for the formation of an electrode array, in accordance with embodiments of the present invention;

FIGS. 5a-d and 6—show a process for forming an electrode array structure, in accordance with embodiments of the present invention;

FIG. 7—shows a block diagram of a plasma enhanced chemical vapour deposition system; and

FIG. 8—shows two linear arrays of electrodes of different widths.

### DETAILED DESCRIPTION

A method of producing an electrode array for a medical implant is provided in which carbon nanotubes are deposited onto electrode contacts at temperatures below the service temperature of a carrier material supporting the electrode contacts. This allows the carrier material, which may be a biocompatible polymer like silicone, to be molded about the electrode contacts before they are coated with carbon nanotubes. By depositing carbon nanotubes onto an electrode contact, the size of the electrode contact may be reduced while maintaining the electrical characteristics of the contact. More electrode contacts may therefore be provided in the array per unit area and in a linear configuration along the length of the array.

The electrode contact may have deposited thereon Carbon Nanotubes (CNTs) by plasma enhanced chemical vapour deposition (PECVD). In other embodiments, the coating of the plurality of electrode contacts with CNTs includes depositing CNTs, typically at around room temperature, using a self-assembled monolayer as a bridging ligand at the CNT-electrode contact interface.

An electrode contact structure for a medical implant is also provided. The electrode contact structure includes an array of spaced apart electrode contacts and a carrier material. Each electrode contact has an exposed portion on which is deposited thereon CNTs. The electrode contacts may have a smaller size in comparison to uncoated platinum electrodes. This may allow an increased density of electrode contacts.

Throughout the following description, the term “electrode array” will be understood to mean a collection of two or more electrodes each comprising an electrode contact and at least one respective conductive pathway such as a conductive filament or wire or strand of conductive filaments or wires. It should be appreciated that in the literature and prior art, the term “electrode array” is sometimes used to refer to the combination of electrode contacts, electrode wires and carrier member in which the electrode contacts and conductive pathways are disposed. The term “conductive pathway” in the context of a pathway to an electrode contact refers to an electrically conductive wire made of a suitable material, for example platinum or Carbon Nanotubes. The term “electrode contact” refers to the element to which the energy is transferred by the conductive pathway, and through which the stimulating energy is applied to the tissue of the implantee.

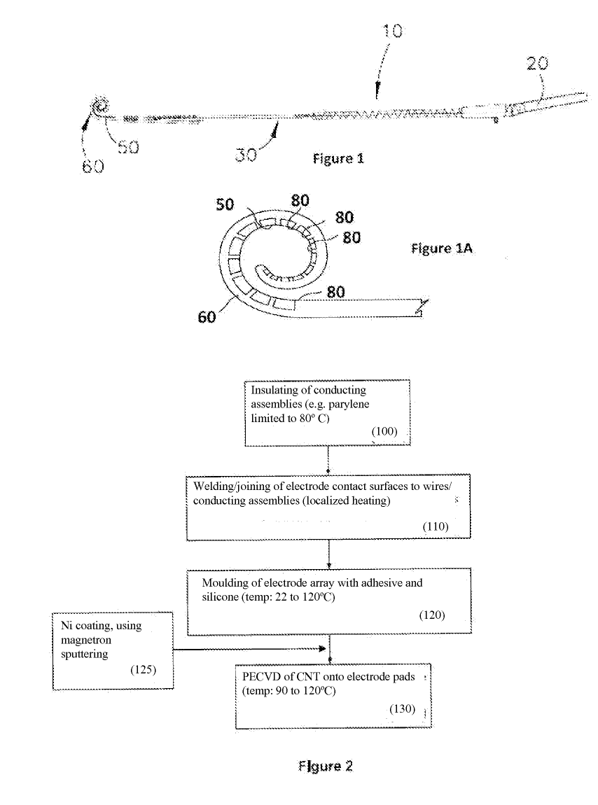

Embodiments of the present invention relate to the manufacture of an electrode array and in particular to an electrode array for use as part of a medical implant. An exemplary medical implant including an electrode array is a cochlear implant of the type shown in FIG. 1. An enlarged view of an embodiment of an electrode array is shown in FIG. 1A.

FIG. 2 is a high-level flow chart of the primary operations performed in one embodiment of a method of producing an electrode array having electrode contacts coated with carbon nanotubes (CNTs).

In step 100, the various conducting assemblies (e.g. electrode conductive pathways) are insulated. The insulating coating is in one form, a biocompatible material with a low dielectric constant such as polytetrafluorethylene (PTFE), Parylene, in particular Parylene C, polyurethane (PU), polyethersulphone (PES), polyphenylsuIphone (PPS), polydimethylsiloxane (PDMS), polyethylene (PE), polyethylene terephthalate (PET) or an amorphous carbon coating. The insulating thickness may range from approximately 0.1 to 5 microns. Application of the insulating coating may be performed by plasma enhanced chemical vapour deposition or another suitable technique.

In step 110, the electrode contacts are connected to the electrode conductive pathways. Conventional electrode conductive pathways in the form of platinum wires in implantable medical devices have a diameter that ranges from approximately 20 to approximately 25 microns. Such conductive pathways can be handled during assembly into an electrode using fine tweezers under a microscope. The conductive surfaces including the surfaces of the electrode contacts and the wires may comprise either a biocompatible conducting material selected from platinum, iridium, palladium, titanium, tantalum, or combinations of thereof or a wire comprising CNTs, for example one or more strands formed by drawing, twisting and spinning CNTs from a CNT forest coated with an insulator to form an insulated cable.

In step 120, the electrode array is over-molded with silicone, at a temperature range of, for example, 22° C. to 120° C. (72 F to 248 F). A silicone adhesive may also be used to assist in maintaining the wires and electrode contacts in position during the over-molding process. In some embodiments, the silicone is cured at room temperature. In other embodiments silicones with a catalyst such as acetoxy or platinum are used, for which curing may be accelerated by heating, for example to about 120° C. (248 F). The over-molded material may alternatively be another suitable polymeric carrier material for the electrode array, for example polyurethane (PU), polydimethylsiloxane (PDMS) or polyetheretherketonc (PEEK). The molding technique used to over-mold the silicone or other polymeric material may, for example, be selected from the group comprising injection moulding, transfer moulding and compression moulding.

After the over-molding process, the electrical stimulating surface of the electrode contacts are cleaned of silicone so as to expose the stimulating surface of each electrode contact in the electrode array. Exposure of the electrical stimulating surface of the electrode contacts may be achieved, for example, by use of a scalpel or laser ablation.

One or more of steps 100, 110 and 120 may be omitted if pre-formed components are received. For example, if the components for the electrode array are received with insulation already applied, then step 100 may be omitted. If the components are also joined into the general structure of an electrode array, but without a carrier, then steps 100 and 110 may be omitted. If the electrode array has been preformed and over-molded with a carrier, then only step 130 may require completion, optionally with step 125 (see herein below). Regardless of which of these options is required, the method involves applying the CNTs to the electrode contacts after the electrode array has been formed with a polymeric carrier material.

In step 130, the electrode contacts are coated with CNTs. In the example represented in FIG. 2 the coating process comprises plasma enhanced chemical vapour deposition. As explained herein below, other low temperature processes for depositing a coating of CNTs onto the electrode contacts may be used.

The coating process is completed at a temperature below the service temperature (the temperature when the material starts to degrade) of the over-molded material. For example, the service temperature for silicone is approximately 250° C. (approximately 482 F) and the service temperature for polyetheretherketone (PEEK) is 350° C. (660 F). The temperature at which the coating process is completed may also be selected having regard to the service temperature of any adhesive used at the wire-to-electrode contact connection. In addition any other components (for example any electronic components) that are present in the assembly being processed that have a service temperature around or less than that of the molding material or adhesive may set the maximum temperature for the deposition process. Accordingly, the coating process, which may be PECVD, is conducted at temperatures below the service temperature of the carrier material, for example at temperatures anywhere in the range from about 250° C. (about 480 F) down to about 120° C. (about 250 F) when silicone is the limiting material or anywhere downwards from about 350° C. (about 660 F) where PEEK is the limiting material. In other embodiments, plasma enhanced chemical vapour deposition is completed at a temperature of between about 90° C. to 120° C. (between about 195 F to 250 F).

In some embodiments, a coating of a catalyst such as nickel ranging from 1 to 100 nm in thickness, more preferably 6+/−3 nm thick is deposited on the electrode contacts by magnetron sputtering (step 125) to assist in reducing the activation energy required for growing the CNTs and consequently reducing the temperature at which they can be grown. The surface of the catalyst can be patterned with features ranging from 60 to 170 nm using e-beam lithography. The deposition of a catalyst such as nickel will not compromise the integrity of the electrode since it is a cold process and can be performed well below the service temperature of the carrier material. Alternatively, catalyst free growth of CNT could be deployed. Prior to vapour deposition, the vacuum chamber may be first filled with an ammonia (electronic grade) and acetylene gas mixture and the samples heated to the operating temperature.

A DC PECVD system may be used to deposit the CNT coating over the exposed stimulating surface of the electrode contacts. As mentioned above, the system may operate at a low temperature, for example 120 C (250 F). The system may operate using parameter settings of a voltage between 200V to 2 kV, preferably 500V to 1300V, a stable discharge current between 10 to 100 mA, typically 30 mA, and a pressure of about 1.5 mbar was maintained for a period of approximately 30 to 45 minutes. In other embodiments, these settings may be varied. For example, the period may range anywhere from 5 minutes to 1 or 2 hours and the pressure in the vacuum chamber may range from 0.2 to 2.2 mbar, preferably 0.5 to 2 mbar.

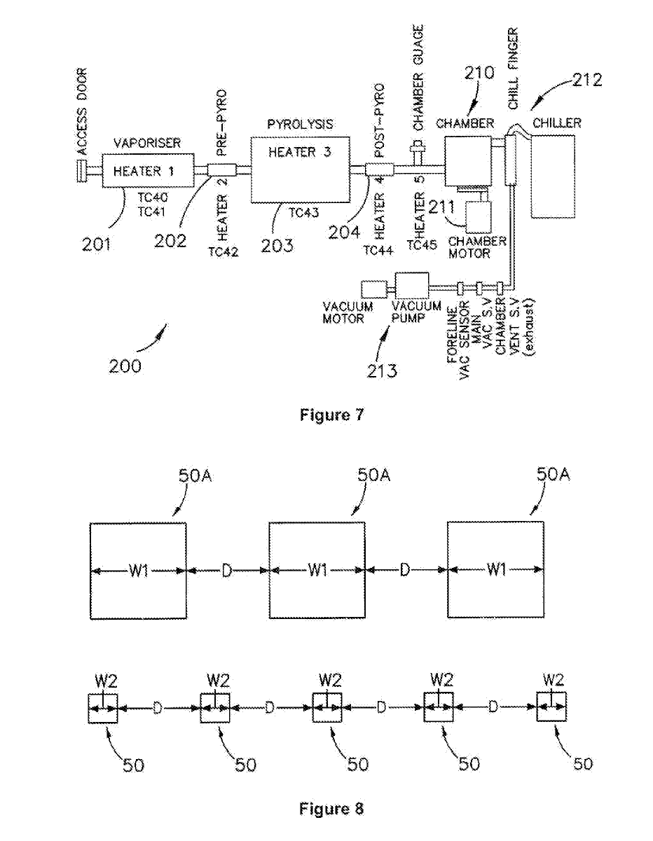

FIG. 7 shows a block diagram of a plasma enhanced chemical vapour deposition system 200 that may be operated using the above parameters. In general, the chemical vapour deposition system 200 includes heaters 201-204 to form the vapour for deposition, a vacuum chamber 210 through which the carbon nanotubes are passed for vapour deposition, a chamber motor 211 for feeding the electrode array through the chamber, a downstream chiller and chill finger 212 (or other suitable heat exchanger) and vacuum pump and motor 213. For PECVD a dc discharge was ignited between a cathode and an anode by applying a voltage typically of 600V in an acetylene/ammonia gas mixture. Acetylene supplies the carbon and ammonia etches away unwanted amorphous carbon produced by the plasma. The ratio of acetylene to ammonia was kept constant during deposition at 1:2 to 1:6 sccm by mass flow controllers. The system includes various inputs, outputs, gauges, sensors and other components, some of which are shown in FIG. 2 and others of which are omitted.

From the foregoing description, it will be appreciated that the use of plasma enhanced chemical vapour deposition allows CNTs to be applied to the electrode contacts at temperatures that will not damage or otherwise compromise the other components in the assembly.

A particular difficulty with the use of silicone and other polymer carriers for an electrode array is that typical techniques for applying CNTs employ temperatures at about 600° C. (about 1100 F) or above. In contrast, and as described above, the service temperature for the carrier material (or insulating polymer or adhesive for the conductive paths) is much lower. It has been discovered that if the CNTs are pre-deposited onto the electrode surfaces using typical high temperature CVD processes in excess of 600° C. before over-molding of the polymer, then the delicate nature of the CNTs means that the step of subsequently over-molding the polymer will tend to damage the CNT coating. The method described above addresses this by using a low-temperature technique for applying CNTs only after the over-molding process has been completed.

Retaining a conductive electrode pad on which the CNTs are grown may provide an advantage of reducing the risk of the CNTs not being electrically connected to each other. This is because CNTs that may not be connected to each other directly, may be electrically connected via conductive electrode pad.

In an alternative to using chemical vapour deposition (i.e. as an alternative to step 130 in FIG. 2), it is possible to coat the electrode contacts with CNTs using self-assembled CNTs grown directly onto the surface of the electrode contact. This process can be conducted at room temperature and therefore can be completed well below the service temperature of the polymeric carrier applied in step 120.

CNTs can be attached to metallic substrates, including Pt, Pt alloys, Ti, Ti alloys, stainless steel and gold, using various techniques including employing a self-assembled monolayer (SAM) as the bridging ligand at the CNT-metal substrate interface. SAMs are created by the chemisorption of hydrophilic “head groups” onto a substrate from either the vapour or liquid phase followed by a slow two-dimensional organization of hydrophobic “tail groups”. The hydrophilic “head groups” assemble together on the substrate, while the hydrophobic tail groups assemble far from the substrate. Areas of close-packed molecules nucleate and grow until the surface of the substrate is covered in a single monolayer.

The CNTs can be functionalized (attached to the 'tail group of the SAM molecule) through wet chemistry processes or in-situ functionalized with reactive functional groups during the CVD process. The attachment reaction of the SAM molecule ‘head group’ is dictated by its molecular structure, examples include alkanethiols and chlorosilanes.

CNTs can be either multi-walled or single walled. In one form, the CNTs used in the present application are single-walled CNTs due to superior electrical properties. However, double-walled CNTs could equally be used depending upon the desired properties for the particular application.

Furthermore, the alignment, purity, continuity, molecular orientation and functionalization of the CNTs also impact the electrical properties. For instance, the use of a catalyst or functional groups may aid the electrical properties. However, these may also adversely affect the biocompatibility of the device. Generally, the absence of functional groups on the CNT will reduce the risk of complications with biocompatibility. However, any combination of these factors may be selected to meet the specific desired functionality depending upon the specific application.

Additionally, the CNTs could be filled with conductive materials for the purpose of optimizing the electrical properties as will be understood by the person skilled in the art.

The following describes one method of completing steps 110 and 120 in the process of manufacturing the electrode array 60, having an electrode wire (forming part of the electrode lead 30) and electrode contacts 50. The method is described with reference to the manufacture of an electrode array 60 that has electrode wires formed from carbon nanotube strands. However, the method may also be applied to electrode wires formed from other materials, for example platinum.

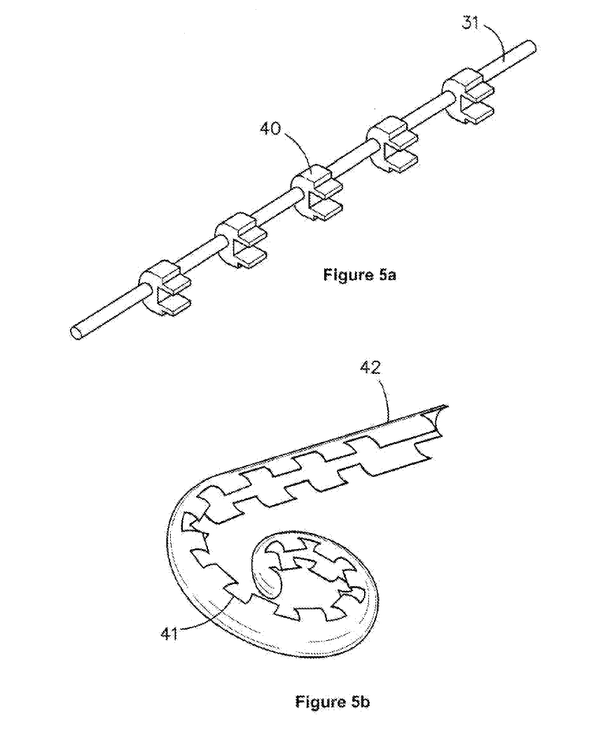

Appropriately-sized platinum electrode contacts are placed in a U-shaped die having a linear configuration. CNT conducting strands are connected to the electrode contacts by, for example welding, tying or by any other suitable method. The CNT conducting strands are exposed at their ends to allow connection with their respective electrode contact, but otherwise include an insulating layer, for example the layer of Parylene C (step 100 in FIG. 2). A droplet of adhesive, such as adhesive silicone is then placed in the trough of each electrode contact to secure the CNT conducting strands. FIG. 4 shows a diagrammatic representation of a U-shaped die 70 containing platinum electrodes 47 each connected to a respective wire 48. The wires 48 form part of the electrode lead 30 shown in FIG. 1. The adhesive silicone is also shown in FIG. 4, referenced 45.

Once all CNT conducting strands have been connected in the U-shaped connecting die 70, a production stylet 44 (if required for the particular implementation), for example a PTFE coated wire, is pressed on top of the CNT conducting strands and adhesive silicone 45 in the troughs of the electrode contacts 47, or otherwise suspended in place in the troughs. The production stylet 44 holds the electrode contacts 47 in a spaced relationship to each other and provides further support to the electrode array. It is removed after the mould has been completed and cured to form a lumen in the lead. The trough of each electrode contact is then partially filled with more adhesive silicone 45. This whole assembly is then placed in an oven to cure the adhesive silicone 45.

The assembly is then removed from the straight die and carefully curved, for example using tweezers under a microscope, to match the shape of a curved moulding die. The assembly is then placed in a curved moulding die with the contacts being located closer to the medial side (inside of the curve). Then, the space in the die is packed with silicone material. A matching die cover is placed over the assembly and pressed down. Alternatively, a closed die can be injection molded with silicone. The die is then placed in an oven to cure the silicone. Once the silicone is cured, the die is then opened to allow the resulting electrode array to be removed from the die.

Silicone covering the electrode contacts is then removed. One technique to achieve this is to cut away the silicone using a scalpel and tweezers under a microscope. In some embodiments the entire electrode array is over-molded with silicone and then cut away in the area of the electrode contacts to expose the electrode contacts. The silicone may also be removed using laser ablation. In other embodiments, the electrode contacts are masked during the moulding process. When a mask is used, the process of manufacture may still include a step of cutting away the silicone, to remove any material that impinged between the mask and the electrode contact and/or to create a smoother edge of the silicone material about the electrode contact.

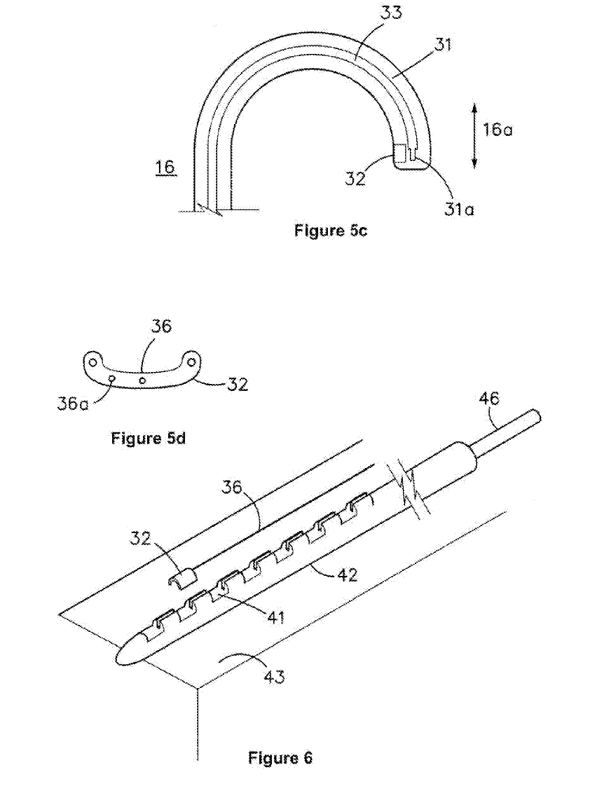

FIGS. 5a-5d and 6 show an alternative method of forming the electrode array 60 (steps 110 and 120 of FIG. 2). First, a production stylet 31 is cut to size from platinum wire coated with polytetrafluoroethylene (“teflon”). A plurality of spacers 40 made of silicone are inserted over the production stylet 31 as seen in FIG. 5a.

At its tip 31A, (FIG. 5c) the teflon coating is removed from the production stylet 31 thereby forming the narrow section at the end of the lumen.

Next, the stylet 31 with the spacers 40 is inserted into a curved die (not shown) and a silicone material is poured into the curved die and cured to form a molded blank 42, which includes the production stylet 31 and the spacers 40 embedded therein. The spacers 40 position the production stylet at a predetermined location within the molded blank 42. The die is shaped so that the blank 42 has an end section 16A that is not curved, but is formed so that it is relatively straight. This section 16A may have a length of about 0.7 mm. The bare tip 31A of the production stylet 31 is disposed in this end section 16A. After the molded blank 42 is cured, it is removed from the die.

The production stylet is then withdrawn from the molded blank 42, leaving behind the lumen 33 having a tip 31A which is narrower then the rest of the lumen. The spacers 40 are also left behind on removal of the production stylet. The non-stick property of the teflon facilitates the easy removal of production stylet 31 from the cured silicone array, thereby leaving a smooth lumen 33 behind. Prior to the removal of the molded blank from the moulding die, the lumen 33 is, of course, circular.

In a separate operation a plurality of electrodes 32 are formed and attached to corresponding wires 36 as follows. First several rings made of platinum are provided. A typical array may have 22 electrodes in which case the following sized rings may be used: 6 rings with an outer diameter of 0.6 mm, 6 rings with an outer diameter of 0.63 mm and 10 rings with an outer diameter of 0.65 mm. It will be appreciated that with an increased number of electrodes as contemplated by the present invention the number and dimensions of the rings will be altered accordingly.

A Parylene-coated Pt/Ir wire is connected to each of the rings as follows. The wire is placed inside the ring and welded, and then the ring is then collapsed and welded, using a welding electrode to form a U-shaped electrode 32 shown in FIG. 5d. The wire 36 extends away from the electrode 32.

Generally, every electrode 32 is connected in this manner to a single wire 36. However, with the technique described above it is relatively easy to connect two or more wires, such as wire 36A, to each electrode 32 as well. Multiple wires provide redundancy in case one of them breaks and may also provide greater mechanical flexibility for a given electrical resistance.

The molded blank 42 is next manually straightened and placed into a production jig 43 adapted to hold the molded blank 42 in the straight configuration (FIG. 6). The production jig comprises a flat piece of chrome-plated brass with indents to hold the electrodes in correct position for attachment to the production stylet. In this configuration a second production stylet 46 is inserted into the lumen 33 to hold the molded blank 42 straight.

The second production stylet 46 is straight and may be made of stainless steel. Once the second production stylet 46 has been fully inserted into the lumen 33, the production jig 43 is removed. Next, each of the electrodes 32 is glued in one of the recesses 41 on the blank 42 as shown in FIG. 5, using a dab of silicone adhesive. Recesses 41 are formed by the shape of the die used to mould the blank. The blank 42 is then backfilled with a silicone filler material and the whole array is cured to complete the array 10. Upon addition of the silicone filler material and curing spacers 40 and the added filler amalgamate to form an homogeneous whole. The second production stylet 46 is then removed.

It will be understood that in other embodiments, the electrode array arrangements need not use a stylet/lumen arrangement, and the various aspects of the invention are equally applicable to non-lumen/stylet arrangements.

To form a cochlear implant, after completion of steps 100 to 130, the electrode array 60 is attached to the cochlear implant receiver/stimulator. The electrode wires in the electrode lead 30 may be joined to the stimulator by crimping, resistance welding, soldering or by dispensing thermally conductive adhesive using jetting. The assembly formed of the electrode array 60 and the receiver/stimulator is packaged and shipped in an electrode array kit that also includes a surgical stylet and, optionally, a straightening jig.

FIG. 1 shows a cochlear implant 10 including a stimulator 20, an electrode lead 30, and having an array of electrodes 60 with corresponding electrode contacts 50. FIG. 1A shows an enlarged view of an electrode 60. The electrode 60 includes a carrier 80 for the electrode contacts, which is formed by the carrier material applied by moulding in step 120 in FIG. 2. In a cochlear implant, this carrier 80 may perform the functions of maintaining the spatial distribution of the electrode contacts 50 and facilitate insertion of the electrode array 60 into the cochlea.

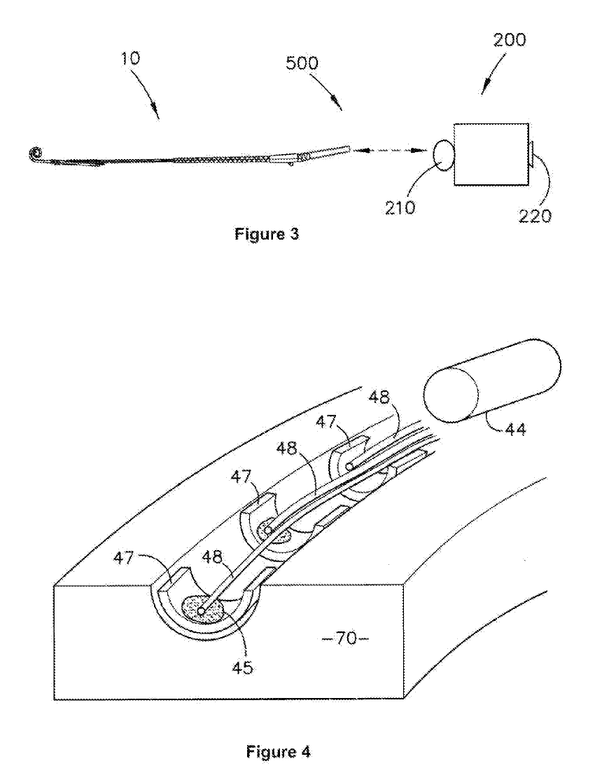

FIG. 3 shows a medical implant system including the cochlear implant 10, in this example, a cochlear implant system 500, comprising an external component 200 (in this case a processor) and the cochlear implant 10. A processor 200 receives input signals in the form of sound information from the surrounding area around the implantee via any suitable means, such as a microphone 220 and processes this data into control signals for transmission to the internal component or stimulator 10. The control signals may be transmitted subcutaneously via transmitting coil 210, to be received by a receiving coil (not shown) of the stimulator 20 of the cochlear implant 10. The control signals are then further processed by circuitry in the stimulator 20 to provide stimulation signals for applying directly to the cochlea of the implantee via electrode array 50 as will be understood by the person skilled in the art.

As noted in the Background above, In the case of cochlear implants, the effectiveness of the implant can be increased if the number of electrode contacts, or stimulating sites, is increased. In order to achieve this, the width of the stimulating element along an electrode array needs to be minimised whilst maintaining effective stimulating surface area. For some electrode arrays, for example where electrodes are spaced laterally and longitudinally in the array, both the width and the length (i.e. the area) of the electrode contact may be relevant measures of electrode contact size that is sought to be reduced.

One way in which the number of electrode contacts can be increased is by decreasing the size of individual electrode contacts. The decreased area occupied by each electrode contact allows a higher number of electrode contacts to be included in the electrode array along the same length, or within the same area. A problem with electrode arrays with electrode contacts of conventional material like platinum is that the smallest possible electrode that can be manufactured has currently effectively reached a limit. As previously described, for a conventional platinum electrode pad, the surface area limit is approximately 0.2-0.3 mm² before charge density exceeds safe limits, resulting in dissolution of the platinum.

It is possible to reduce the overall size of an electrode contact by increasing its effective surface area. Using the method described above with reference to FIG. 2 (including any of the alternatives described herein above), the effective surface area of an electrode contact in the electrode array 60 is increased by coating the surface of the electrode contact with Carbon Nanotubes (CNTs). This enables electrode pads, and in particular platinum electrode pads, to have a surface area of less than 0.2-0.3 mm² without exceeding charge density limits.

The deposition of CNT coatings on Pt electrode contact surfaces enables a reduction in their width up to about 3 orders of magnitude while preserving useful impedance characteristics for at least some medical implant applications. The minimum size limit will depend on the application and the requirements specification for the particular device being manufactured. Embodiments of the invention may be used to produce electrode contacts of width from about 0.34 mm down to about 0.00035 mm (1.225×10⁻⁷ mm² surface area for a square electrode contact). Electrode contact widths within ranges of about 0.30 mm to about 0.03 mm, about 0.03 mm to about 0.003 mm and 0.003 mm to about 0.0003 mm may alternatively be produced. The length of the electrode contacts may have a similar range of magnitudes, recognising that electrode contacts may be square, rectangular, circular, U-shaped or another shape.

The spacing between the electrode contacts in the array is selected depending on the requirements for the particular application. A limiting factor may be the dielectric constant of the carrier material and/or any additional material that may be disposed between the electrode contacts during the over-moulding process (step 120 of FIG. 2). In other words, the limiting factor may be the required impedance between adjacent electrode contacts.

Some cochlear devices may require a minimum level of isolation between receptive areas, which leads to another limiting factor on the minimum separation of the electrode contacts from each other. Despite this, if the electrode contacts are reduced in size, then they occupy a smaller area, allowing increased space between each electrode contact for the same number of electrode contacts per unit length. Accordingly, the process of coating electrode contacts with CNTs described herein can be used to increase the ratio of the distance between electrode contacts to the width of the electrode contacts within the design objectives/constraints of a particular number of electrode contacts per unit length (typically higher than that for devices with existing uncoated platinum electrodes) and sufficient isolation between electrode pads (typically substantially constant for electrodes at the same distance from the tissue to be excited). Isolation between receptive areas can also be maintained while reducing the distance between the receptive areas by minimising the distance between the charge emitter (electrode contact) and the receptor.

By way of example, taking into account the requirements for the number of electrode contacts, minimum size of the electrode contacts and required isolation between electrode contacts in a cochlear implant, the maximum ratio of the distance between pads to the width of pads using platinum (Pt) electrode contacts is currently about 0.857:1. The current limitation of conventional Pt electrode contacts is that a minimum contact width of 0.35 mm is required in order to achieve the required impedance levels and the smallest distance between pads to maintain sufficient isolation between them is about 0.3 mm.

The ratio of the distance between pads to the width of the pads may be greater than about 0.857. In various embodiments electrode arrays may be produced with distance-to-width ratios of about 0.900, from about 1.000 to about 2.000, from about 2.100 to about 5.100, from about 5.200 to about 10.000, from about 10.000 to about 50.000, from about 50 to about 100, from about 100 to about 500, from about 500 to about 880, or even greater. The electrode contacts may have corresponding ratios of distance between contacts to the length of the contacts when formed into a two-dimensional array.

FIG. 8 shows two linear electrode arrays. One array includes electrode contacts 50A with a width W1. The electrode contacts 50A may, for example, be conventional Pt electrode contacts. The other array includes electrode contacts 50 with a smaller width W2, which may be Pt with a CNT coating. FIG. 8 illustrates the increased ratio of the distance (D) between the electrode contacts to the width of the electrode contacts achievable as a result of using smaller electrode contacts. In the example shown in FIG. 8, the distance (D) has been maintained the same in both arrays which, for the size reduction illustrated, allows five of the smaller electrodes to be located in the linear space previously occupied by three of the larger electrodes.

It will be appreciated that in applications where the distance (D) does not need to be maintained, then many more electrode contacts per unit area or per unit length may be included in an electrode array. These applications may include cochlear implants, with acceptance of the lack of total isolation of receptive areas to one electrode contact. In other words, the electrode contacts can be placed closer together if it is accepted that a single receptive area may be stimulated by the electrode contact closest to it as well as one or more other electrode contacts in proximity to it. Alternatively, the distance D can be reduced and a larger quantity of electrode contacts may be included in the electrode array.

Smaller stimulating pads can also facilitate the use of measuring electrodes in the electrode array as there is more space to include them. The additional electrode pads can also be used as earth or return electrodes enabling the use of alternative neural stimulation paradigms.

The method of manufacture of an electrode array may also be applied to other types of medical implants such as Auditory Brainstem Implants (ABIs), Functional Electrical Stimulation implants (FESI's), and Spinal Cord Stimulators (SCSs).

Further features and advantages of the present invention are described in U.S. application Ser. No. 12/743,804, filed on May 19, 2010, entitled “ELECTRODE ARRAY FOR A COCHLEAR IMPLANT,” which is a National Stage Application of International Application No. PCT/AU2008/001718, filed Nov. 19, 2008, entitled “ELECTRODE ARRAY FOR A COCHLEAR IMPLANT,” which claims priority from Australian Provisional Patent Application No. 2007906334, filed Nov. 19, 2007. The contents of these applications are hereby incorporated by reference herein.

Further features and advantages of the present invention are described in Australian Provisional Application No. 2009903424 filed on Jul. 22, 2009. The contents of this application is hereby incorporated by reference herein.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All patents and publications discussed herein are incorporated in their entirety by reference thereto.

What is claimed is:

1. A method of manufacturing an electrode array for a medical implant, comprising:
providing a plurality of spaced apart electrode contacts supported by a biocompatible polymeric carrier material extending around the contacts, leaving at least a portion of each of the plurality of electrode contacts exposed, each electrode contact being connected to a respective conductive path extending from the electrode contact; and
depositing carbon nanotubes on the exposed portion of one or more of the plurality of electrode contacts at a temperature less than a service temperature of the polymeric carrier material.

2. The method of claim 1, wherein the carbon nanotubes are deposited on the exposed portion of the one or more electrode contacts using plasma enhanced chemical vapour deposition.

3. The method of claim 2, further comprising:
coating the exposed portion of the one or more electrode contacts with a catalyst for encouraging the growth of carbon nanotubes prior to depositing the carbon nanotubes.

4. The method of claim 3, wherein the catalyst is nickel, and is coated on the electrode contacts at a thickness ranging from about 1 to 100 nm using a cold deposition technique.

5. The method of claim 2, wherein the process of providing carbon nanotubes on the exposed portion of each of the plurality of electrode contacts is carried out at a temperature between about 190 F (90° C.) and 660 F (350° C.).

6. The method of claim 2, wherein depositing carbon nanotubes on the exposed portion of one or more of the plurality of electrode contacts is carried out at a temperature between about 250 F (120° C.) and 480 F (250° C.).

7. The method of claim 1, wherein providing the plurality of electrode contacts with carbon nanotubes comprises depositing carbon nanotubes using a self-assembled monolayer as a bridging ligand at the carbon nanotube-electrode contact interface.

8. The method of claim 1, further comprising:
joining each of the electrode contacts to at least one of the respective conductive paths;
positioning the joined electrodes in a spaced relationship;
over-molding the polymeric carrier material around the joined electrodes; and
exposing at least a portion of each of the electrode contacts prior to deposition of the carbon nanotubes.

9. The method of claim 1, wherein the polymeric carrier material is selected from a group comprising silicone, polyurethane, polydimethylsiloxane and polyetheretherketone.

10. The method of claim 1, wherein the electrode contacts are formed from platinum and have an exposed portion with a surface area of less than approximately 0.04 mm2.

11. The method of claim 1 wherein the electrode contacts have a maximum width of approximately 0.35 mm and the distance between at least two of the electrode contacts is approximately 0.3 mm or less.

12. An electrode array for a medical implant, the electrode array comprising a plurality of spaced apart electrode contacts that each have an exposed portion on which is deposited carbon nanotubes, each contact being connected to at least one conductive path, and a biocompatible carrier material supporting the conductive paths and electrode contacts and extending around the exposed portions of the electrode contacts.

13. The electrode array of claim 12, wherein the carrier material has a service temperature of about 660 F (350° C.) or less.

14. The electrode array of claim 13, wherein the carrier material has a service temperature of about 480 F (250° C.) or less.

15. The electrode array of claim 12, wherein the CNTs are deposited on the exposed portion by plasma enhanced chemical vapour deposition.

16. The electrode array of claim 12 wherein the electrode contacts have a width of between about 0.34 mm to about 0.00035 mm, and the distance between at least two adjacent electrode contacts is about 0.3 mm or less.

17. A medical implant system comprising:
an external component for receiving an input signal and for processing the input signal to provide a control signal
an internal component for receiving the control signal and for converting the control signal into a stimulation signal and applying the stimulation signal to tissue of an implantee via a plurality of spaced apart electrode contacts connected to the internal component via a plurality of corresponding conductive paths, with a carrier material extending between and supporting each of the electrode contacts, wherein each electrode contact has an exposed portion on which is deposited carbon nanotubes.

18. The medical implant system of claim 17, wherein the ratio of the distance between the electrode contacts and the width of the electrode contacts is at least 10.

19. The medical implant system of claim 17, wherein the ratio of the distance between the electrode contacts and the width of the electrode contacts is at least 100.

20. The medical implant system of claim 17, wherein the medical implant is a cochlear implant, the external component is a processor and the internal component is a stimulator.
